(19) United States
(12) Plant Patent Application Publication
(10) Pub. No.: US 2024/0298557 P1
(43) Pub. Date: Sep. 5, 2024

(54) Strawberry Plant Named 'Fukushima ST14Go'

(50) ;

(71) Applicant: Fukushima Prefecture, Koriyama City (JP)

(72) Inventors: Miho Akiba, Koriyama City (JP); Satomi Miyashita, Shibukawa City (JP)

(21) Appl. No.: 18/445,019

(22) Filed: Mar. 3, 2023

(51) Int. Cl. A01H 6/74

(52) U.S. Cl. CPC A01H 6/7409
USPC PLT/208

(57) ABSTRACT

‘Fukushima ST14Go’ is a new vigorous strawberry variety having an upright growth habit that can withstand cold temperatures. ‘Fukushima ST14Go’ has large, strong yellowish red, conical fruit having pale pink flesh.

Genus and species: The strawberry plant of this invention is botanically known as Fragaria ananassa L.

Variety denomination: The variety denomination is ‘Fukushima ST14Go’.

### BACKGROUND

‘Fukushima ST14Go’ arose from a cross between maternal variety ‘Kaorino’ and paternal variety ‘Tochiotome’ performed in Koriyama city, Fukushima prefecture, Japan in 2012. The plant was first asexually reproduced in Koriyama city, Fukushima prefecture by runners. ‘Fukushima ST14Go’ was selected from 162 individuals in 2014. The characteristics of ‘Fukushima ST14Go’ are stable over subsequent asexual reproduction by runners.

Field tests were conducted in six locations (Date City, Sukagawa City (2 locations), Kagamiishi Machi, Tanagura Machi, and Iwaki City) in 2018; four locations (Sukagawa City (2 locations), Aizuwakamatsu City and Iwaki City) in 2019, three locations (Aizuwakamatsu City, Okuma Machi, and Iwaki City) in 2020 and seven locations (Date City, Tanagura Machi, Aizuwakamatsu City (2 locations), Soma City, Okuma Machi, and Iwaki City) in 2021.

### BRIEF SUMMARY

‘Fukushima ST14Go’ is maintained by asexual propagation by runners in Fukushima, Japan. ‘Fukushima ST14Go’ has an upright growth habit; and large, strong yellowish red, conical fruit that has pale pink flesh. The type of bearing is non-remontant. ‘Fukushima ST14Go’ can be distinguished from parent varieties ‘Kaorino’ (unpatented) and ‘Tochiotome’ (unpatented) as shown in Table 1. ‘Tochiotome’ is a close comparison variety.

TABLE 1
Comparison of ‘Fukushima ST14Go’ to Parental Cultivars.
Colors are designated with reference to The
Royal Horticultural Society (RHS) Colour Chart.

| Name of | | | Characteristic |
|---|---|---|---|
| Similar | | Characteristic of | of Variety |
| Variety | Characteristics | Similar Variety | of Application |
| ‘Kaorino’ | Plant: vigor | moderately | strong |
| | relative intensity | strong | darker than |
| | of color of upper | | ‘Kaorino’ |
| | side of leaf | | |
| | Fruit: color of core | White 155-B | light red 33-C |
| ‘Tochiotome’ | Plant: growth habit | semi-upright | upright |
| | Fruit: color | brilliant red | strong yellowish |
| | | 45-A | red 44-A |
| | Fruit: position | deep below | level with |
| | of achenes | surface | surface |

### BRIEF DESCRIPTION OF THE DRAWINGS

The colors in the photograph are depicted as nearly true as is reasonably possible to obtain in color reproductions of this type. The photographs were obtained from plants of less than one year of age. Seedlings were started in July, planted in September and grown until April.

FIG. 1 depicts plants of ‘Fukushima ST14Go’ grown in a glass greenhouse in Fukushima, Japan.

FIG. 2 shows exterior and interior of representative fruit of ‘Fukushima ST14Go’.

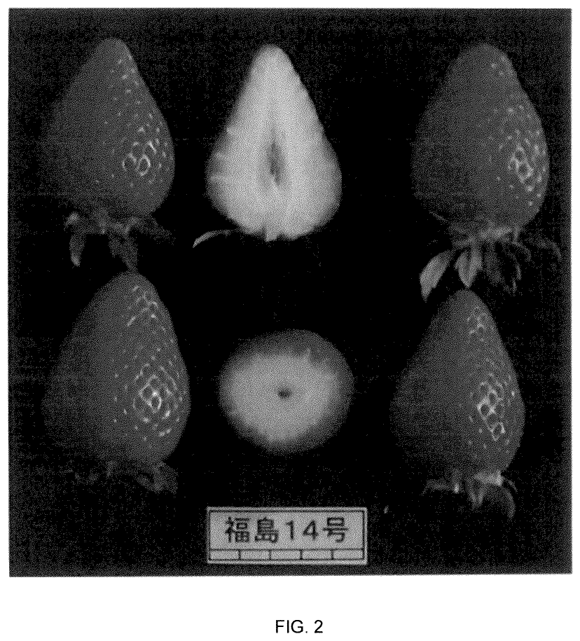

FIG. 3 depicts plants of comparison variety ‘Tochiotome’ grown in a glass greenhouse in Fukushima, Japan.

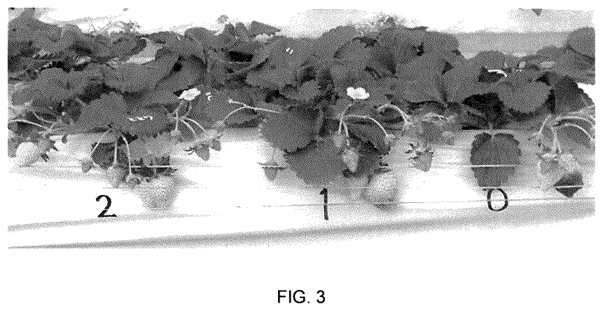

FIG. 4 shows exterior and interior of representative fruit of comparison variety ‘Tochiotome’.

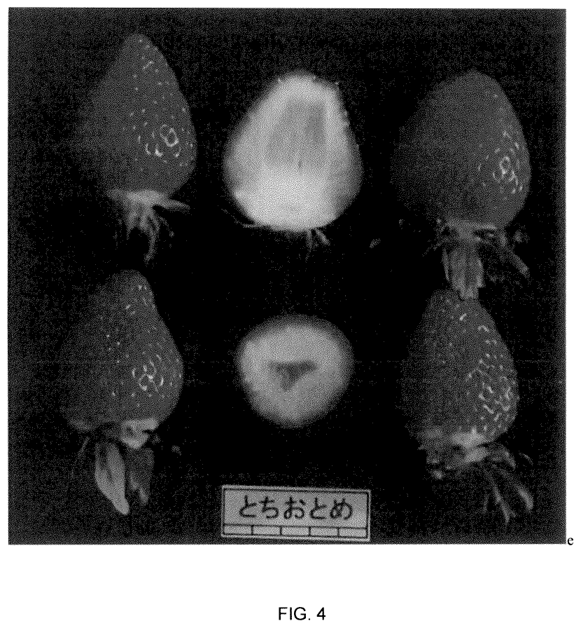

FIG. 5 shows exterior and interior of representative fruit of comparison variety ‘Kaorino’.

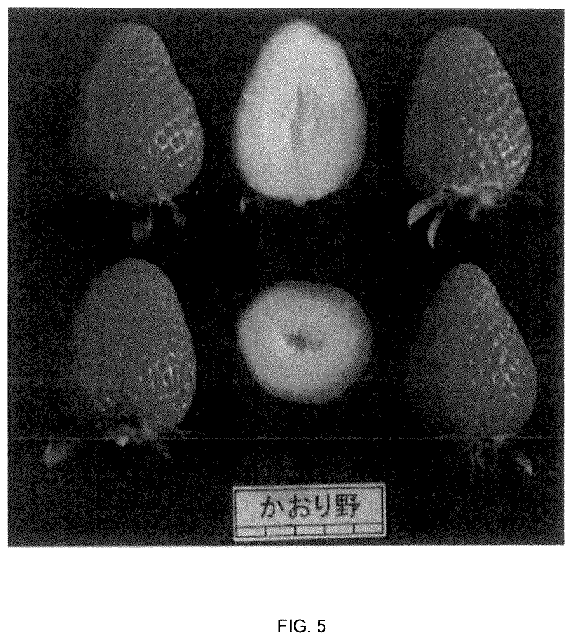

### DETAILED DESCRIPTION

### Characteristics of ‘Fukushima ST14Go’

The plant growth habit is upright. The plant vigor is strong and is maintained without lighting even in the severe cold period (Table 2). The generation of side buds is rare and the fruit cluster is slightly long and hard to break, so the work efficiency is excellent.

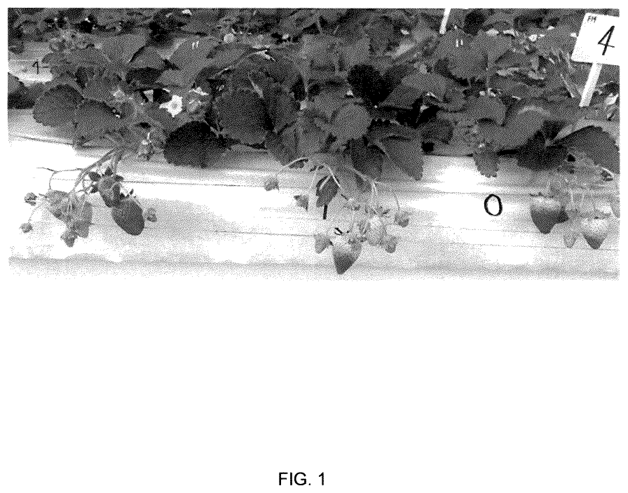

The fruit shape is conical, and the fruit color is strong yellowish red (FIG. 1, Table 7). Fruit are long and narrow, which is the same as ‘Tochiotome’. The following cahracteristics of ‘Fukushima ST14Go’ fruit are also the same as ‘Tochiotome’: ‘Fukushima ST14Go’ exhibits medium adhesion of calyx to fruit; the position of the calyx in relation to the fruit is flat; and the color uniformity is weak. The stalk length of ‘Fukushima ST14Go’ is 25.9 cm compared to 21.9 cm for ‘Tochiotome’.

‘Fukushima ST14Go’ has 4.5 stolons compared to 3.9 stolons for ‘Tochiotome’.

‘Fukushima ST14Go’ has the following foliage characteristics compared to ‘Tochiotome’. The length is 7.2 cm compared to 6.0 cm for ‘Tochiotome’; the length and width of apical lobes is 1.28 compared to 1.24 for ‘Tochiotome’; and the leaf area is 107.2 cm² compared to 82.0 cm² for ‘Tochiotome’. The basal shape, acute, is the same as ‘Tochiotome’. Both cultivars have medium surface roughness and strong surface gloss. Both also have serration of the tip; and a transverse section shape that is curved upward. Petioles are 9.1 cm compared to 5.6 cm for ‘Tochiotome’.

Flowers of ‘Fukushima ST14Go’ have 5 petals and are of medium diameter, both of which are the same as ‘Tochiotome’.

The root development for ‘Fukushima ST14Go’ was generally better than that of ‘Tochiotome’. When seedlings were placed in water for three weeks, the number of roots for ‘Fukushima ST14Go’ was 13.9 compared to 12.9 for ‘Tochiotome’; the maximum root length was 5.1 cm for ‘Fukushima ST14Go’ compared to 4.4 cm for ‘Tochiotome’; and the roots of ‘Fukushima ST14Go’ were finer than those of ‘Tochiotome’.

Harvesting starts early and yields are high by the end of the year. The percentage of large fruits is high and there are few malformed fruits. The taste is good, the sugar content is high, and skin firmness is high (Tables 4 to 7).

### Evaluation in Field Tests

Field tests provided the following additional characteristics. The plant vigor was maintained even in the severe cold period (common). Yields were high (Sukagawa). The taste was good with little variation in taste from season to season. Damage was not noticeable after harvest. The tail fruit of each fruit cluster appeared small.

In view of the vigor of the variety, it is expected that ‘Fukushima ST14Go’ can be cultivated throughout the Fukushima prefecture as well as corresponding areas that have similar cold periods.

The development of clusters may be delayed after the second cluster is harvested. The top fruit is large, but bottom fruit tends to become smaller.

‘Fukushima ST14Go’ has moderate to weak resistance to Antrhacnose and weak resistance to chlorosis. Pest control can be carried out in the same manner as the practice for ‘Tochiotome’.

Fruit hardiness and pulp hardness decreased more than that of ‘Tochiotome’ 2 and 4 days after harvest, and post-harvest storability was inferior to that of ‘Tochiotome’.

TABLE 2
Comparison of ‘Fukushima ST14Go’ and parental cultivar growth
characteristics at start of harvest (December 2020, without lighting,
Fukushima) of plants less than one year of age. Seedling were
started in July, planted in September, and grown until April.

| | Plant | | Plant | Petiole | Leaflet | Terminal |
|---|---|---|---|---|---|---|
| | growth | Plant | height | length | length | inflorescence |
| Variety | habit | vigor | (cm) | (cm) | (cm) | length (cm) |
| ‘Fukushima | upright | strong | 23.1 | 9.1 | 7.2 | 25.9 |
| ST14Go’ | | | | | | |
| ‘Tochiotome’ | semi- | medium | 14.1 | 5.6 | 6.0 | 21.9 |
| (standard) | upright | | | | | |
| ‘Kaorino’ | upright | strong | 23.4 | 11.1 | 7.9 | 39.9 |
| (reference) | | | | | | |

TABLE 3
Comparison of ‘Fukushima ST14Go’ and parental cultivar flowering
dand harvest dates (November 2020 to May 2021, Fukushima)

| | Terminal inflorescence | | | Second inflorescence | | |
|---|---|---|---|---|---|---|
| | Flowering | Harvest | No. of | Flowering | Harvest | No. of |
| Variety | date | date | flowers | date | date | flowers |
| ‘Fukushima | November 10 | December 17 | 14.1 | December 20 | February 1 | 11.4 |
| ST14Go’ | | | | | | |
| ‘Tochiotome’ | November 14 | December 28 | 15.7 | January 5 | February 23 | 11.5 |
| (standard) | | | | | | |
| ‘Kaorino’ | November 4 | December 10 | 12.9 | December 6 | January 19 | 13.0 |
| (reference) | | | | | | |
| | Third inflorescence | | | Fourth inflorescence | | |
| | Flowering | Harvest | No. of | Flowering | Harvest | No. of |
| Variety | date | date | flowers | date | date | flowers |
| ‘Fukushima | February 27 | March 31 | 7.1 | April 13 | May 11 | 10.0 |
| ST14Go’ | | | | | | |
| ‘Tochiotome’ | March 15 | April 19 | 8.0 | April 14 | May 16 | 9.6 |
| (standard) | | | | | | |
| ‘Kaorino’ | February 9 | March 16 | 8.1 | March 5 | April 8 | 7.1 |
| (reference) | | | | | | |

TABLE 4
Comparison of ‘Fukushima ST14Go’ and parental cultivar fruit production
by month¹ (November 2020 to May 2021, Fukushima)

| | November | | December | | January | | February | | March | | April | | May | | Total¹⁾ | |
|---|---|---|---|---|---|---|---|---|---|---|---|---|---|---|---|---|
| | | Yield | | Yield | | Yield | | Yield | | Yield | | Yield | | Yield | | Yield |
| Variety | No. | (g) | No. | (g) | No. | (g) | No. | (g) | No. | (g) | No. | (g) | No. | (g) | No. | (g) |
| ‘Fukushima ST14Go’ | 0.1 | 1.6 | 3.5 | 88.8 | 7.0 | 109.4 | 5.8 | 106.3 | 2.7 | 45.7 | 5.2 | 81.2 | 5.4 | 105.6 | 29.7 | 538.7 |
| ‘Tochiotome’ | 0.0 | 0.0 | 0.7 | 24.6 | 7.4 | 130.8 | 3.5 | 67.3 | 4.1 | 63.5 | 6.9 | 119.1 | 4.7 | 95.5 | 27.3 | 500.6 |
| (standard) | | | | | | | | | | | | | | | | |
| ‘Kaorino’ (reference) | 0.0 | 0.0 | 3.2 | 98.0 | 7.3 | 124.1 | 5.6 | 95.0 | 2.8 | 67.3 | 7.6 | 118.8 | 7.1 | 105.8 | 33.6 | 609.0 |

¹⁾fruit of 6.0 g or greater (excluding malformed fruit)

TABLE 5
Comparison of ‘Fukushima ST14Go’ and parental cultivar yield characteristics
per plant (November 2020 to May 2021, Fukushima)

| | On-specification yield¹⁾ | | | | | | Off-specification yield | | | | | | | |
|---|---|---|---|---|---|---|---|---|---|---|---|---|---|---|
| | 3L | | 2L | | L | | M/S | | Malformed | | Offal | | | |
| | (≥25 g) | | (≥17 g) | | (≥12 g) | | (≥6 g) | | fruit²⁾ | | fruit³⁾ | | Total | |
| | | Yield | | Yield | | Yield | | Yield | | Yield | | Yield | | Yield |
| Variety | No. | (g) | No. | (g) | No. | (g) | No. | (g) | No. | (g) | No. | (g) | No. | (g) |
| ‘Fukushima | 6.9 | 230.9 | 6.8 | 138.5 | 5.4 | 77.1 | 10.7 | 92.2 | 1.2 | 21.0 | 4.4 | 20.2 | 35.3 | 579.9 |
| ST14Go’ | | | | | | | | | | | | | | |
| ‘Tochiotome’ | 5.6 | 190.0 | 6.7 | 139.9 | 6.6 | 94.8 | 8.4 | 76.0 | 2.9 | 67.9 | 2.6 | 10.9 | 32.8 | 579.4 |
| (standard) | | | | | | | | | | | | | | |
| ‘Kaorino’ | 7.7 | 263.6 | 7.4 | 153.9 | 6.1 | 87.1 | 12.3 | 104.4 | 4.2 | 83.6 | 8.1 | 32.9 | 45.9 | 725.5 |
| (reference) | | | | | | | | | | | | | | |

¹⁾fruit of 6.0 g or greater (excluding malformed fruit)
²⁾malformed fruit of 6.0 g or greater
³⁾fruit of less than 6.0 g

TABLE 6
Comparison of ‘Fukushima ST14Go’ and parental cultivar fruit production characteristics (November 2020 to May 2021, Fukushima).

| | | | Irregular | | | | | | | | | | | | | | |
|---|---|---|---|---|---|---|---|---|---|---|---|---|---|---|---|---|---|
| | Avg. | Product | shaped | | | | | | | | | | | | | | |
| | fruit | fruit | fruit | Fruit firmness⁴⁾ (gf) | | | | | | | | | | | | | |
| | weight¹ | ratio²⁾ | ratio³⁾ | December | | January | | February | | March | | April | | May | | Avg. | |
| Variety | (g) | (%) | (%) | Skin | Flesh | Skin | Flesh | Skin | Flesh | Skin | Flesh | Skin | Flesh | Skin | Flesh | Skin | Flesh |
| ‘Fukushima | 18.2 | 84.1 | 4.8 | 105.0 | 22.9 | 140.4 | 52.0 | 125.6 | 36.2 | 148.9 | 43.8 | 106.6 | 48.0 | 91.9 | 32.4 | 119.7 | 39.2 |
| ST14Go’ | | | | | | | | | | | | | | | | | |
| ‘Tochiotome’ | 18.3 | 83.4 | 4.6 | 151.9 | 38.1 | 118.5 | 55.5 | 118.7 | 40.7 | 124.6 | 60.3 | 84.8 | 36.6 | 65.9 | 21.5 | 110.7 | 42.1 |
| (standard) | | | | | | | | | | | | | | | | | |
| ‘Kaorino’ | 17.8 | 71.5 | 8.8 | 97.0 | 38.5 | 103.3 | 32.2 | 124.7 | 31.9 | 152.4 | 54.6 | 87.9 | 28.3 | 82.3 | 33.9 | 107.9 | 36.6 |
| (reference) | | | | | | | | | | | | | | | | | |

¹Product fruit weight/product fruit number
²⁾Product fruit number/total fruit number × 100
³⁾Irregular shaped fruit number/product fruit number × 100 (*product fruit: fruit of 6.0 g or greater excluding malformed fruit)
⁴⁾measured by a creepmeter (YAMADEN, RE2-3305S), penetration resistance to a 3 mm dia. cylindrical plunger penetrated at a maximum rate of 1 mm/sec.

TABLE 7
Comparison of ‘Fukushima ST14Go’ and parental cultivar fruit quality characteristics (November 2020 to May 2021,
Fukushima). Colors are designated with reference to The Royal Horticultural Society (RHS) Colour Chart.

| | Fruit | Skin | Sugar content²⁾ (% Brix) | | | | | | Acidity²⁾ (%) | | | | | |
|---|---|---|---|---|---|---|---|---|---|---|---|---|---|---|
| Variety | shape¹⁾ | color¹⁾ | December | January | February | March | April | May | December | January | February | March | April | May |
| ‘Fukushima | conical | strong | 11.7 | 9.3 | 11.2 | 10.6 | 12.1 | 10.8 | 0.49 | 0.41 | 0.48 | 0.47 | 0.63 | 0.68 |
| ST14Go’ | | yellowish | | | | | | | | | | | | |
| | | red 44A | | | | | | | | | | | | |
| ‘Tochiotome’ | conical | vivid red | 11.0 | 9.3 | 10.9 | 11.2 | 10.3 | 9.6 | 0.53 | 0.43 | 0.47 | 0.50 | 0.60 | 0.69 |
| (standard) | | 45A | | | | | | | | | | | | |
| ‘Kaorino’ | conical | strong | 9.2 | 9.1 | 10.6 | 11.1 | 9.3 | 10.2 | 0.42 | 0.34 | 0.44 | 0.45 | 0.48 | 0.56 |
| (reference) | | yellowish | | | | | | | | | | | | |
| | | red 44A | | | | | | | | | | | | |

¹⁾at the time of investigation in January.
²⁾measured by a strawberry Brix meter (ATAGO. PAL-BX/ACID4)

What is claimed is:

1. A new and distinct cultivar of strawberry plant substantially as described and illustrated herein.
